(19) United States
(12) Patent Application Publication
(10) Pub. No.: US 2021/0121077 A1
(43) Pub. Date: Apr. 29, 2021

(54) PHYSICAL PARAMETER MEASURING DEVICES

(71) Applicants: Randall J. LEE, Woodside, CA (US); Allan W. MAY, Woodside, CA (US)

(72) Inventors: Randall J. LEE, Woodside, CA (US); Allan W. MAY, Woodside, CA (US)

(21) Appl. No.: 17/140,871

(22) Filed: Jan. 4, 2021

Related U.S. Application Data
(63) Continuation of application No. PCT/US19/40637, filed on Jul. 3, 2019.
(60) Provisional application No. 62/694,362, filed on Jul. 5, 2018.

(51) Int. Cl. A61B 5/0205; A61B 5/00; A61B 5/318; A61B 5/28; A61B 5/053; A61B 5/332; A61B 5/145; A61B 5/11; A61B 7/00

(52) U.S. Cl. CPC A61B 5/0205; A61B 5/7267; A61B 5/318; A61B 5/28; A61B 5/0022; A61B 5/053; A61B 5/087; A61B 5/0006; A61B 5/4504; A61B 5/14532; A61B 5/1112; A61B 7/00; A61B 2562/0219; A61B 5/332

(57) ABSTRACT

Described herein are devices, systems, and methods for sensing a physical parameter of an individual. In some embodiments, a physical parameter of an individual comprises an ECG sensed from the individual. In some embodiments, a physical parameter comprises a heart rate of an individual. In some embodiments, a physical parameter comprises a blood pressure of an individual. Generally, devices, systems, and methods described herein for physical parameter measurement include an application specific integrated circuit configured to facilitate effective physical parameter measurement.

### CROSS-REFERENCE

This application is a continuation application of International Application No. PCT/US2019/040637, filed Jul. 3, 2019, which claims the benefit of U.S. Provisional Application No. 62/694,362, filed Jul. 5, 2018, which application is incorporated herein by reference.

### BACKGROUND

Monitoring patient parameters such as, for example, vital signs is useful (and frequently critical for) adequately treating patients and preventing disease.

For example, cardiac disease is prevalent worldwide and includes many different forms of disease including, for example, coronary artery disease, arrhythmia, and heart failure. Monitoring cardiac function is critical for diagnosing and treating heart disease.

### SUMMARY

Described herein are devices, systems, and methods configured to measure certain physical parameters including the monitoring of the cardiac function of an individual.

In some embodiments, a device, system, or method as described herein is further configured to analyze cardiovascular data sensed from an individual and determine a diagnosis.

In some embodiments, a device, system, or method as described herein is further configured to analyze cardiovascular data sensed from an individual and determine a prognosis.

In some embodiments, a device, system, or method as described herein is further configured to analyze cardiovascular data sensed from an individual and determine a likelihood of an occurrence of an adverse event associated with said cardiovascular data.

In some embodiments, a device as described herein comprises a handheld computing device comprising one or more electrodes configured to sense an ECG of an individual when the individual contacts the one or more electrodes with a skin surface. For example, in some embodiments, a device as described herein comprises an ECG monitoring device configured to sense an ECG of an individual when the individual contacts a first electrode of the device with a first hand and contacts a second electrode of the device with a second hand. In these embodiments, when a left hand contacts the first electrode of the device and a right hand contacts a second electrode of the device a lead I is sensed, wherein a lead I comprises a difference in electrical potential between the left and right hands (as measured over time). In some embodiments, a device as described herein further comprises a display, which, in some embodiments, is configured to display a sensed ECG. In some embodiments, a device as described herein comprises a user interface which may be either a manually controlled interface or a graphic user interface (e.g. a touch-screen controlled interface).

A device as described herein, typically, includes a microprocessor that, in some embodiments, is configured to execute instructions such as, for example, computer readable code. In some embodiments of the device that include a microprocessor, the microprocessor is configured to cause the transmission of a signal including data from a transmitter associated with the device. In some embodiments of the device that include a microprocessor, the microprocessor is configured to activate a sensor that is otherwise in a dormant or temporarily non-functional state (e.g. for power saving purposes) so that the activated sensor senses a cardiovascular parameter of an individual. In some embodiments of the device that includes a microprocessor, the microprocessor is configured to analyze a sensed cardiovascular parameter such as, for example, analyzing a sensed ECG and determining whether the ECG is normal or, for example, the sensed ECG shows that an arrhythmia is present.

In some embodiments, a system as described herein comprises a device for sensing a cardiovascular parameter of an individual such as, for example, an ECG monitoring device as described herein that interfaces with one or more devices or systems (including cloud computing systems) that are either remote from the device and/or are not directly hardwired to the device (e.g. wireless components). In some embodiments, a system as described herein comprises a computing device configured to wirelessly couple with an integrated sensing device that, in some embodiments, comprises an application specific integrated circuit.

In some embodiments, a device, system, or method as described herein is an application specific integrated circuit configured for use with a physical parameter measuring device. An application specific integrated circuit configured for use with a physical parameter measuring device as described herein is configured to contain multiple components on a single substrate. For example, in some embodiments, an application specific integrated circuit configured for use with a physical parameter measuring device comprises a single substrate that includes one or more of a microprocessor, a transmitter, a receiver, one or more sensors, and a power source.

Embodiments of an application specific integrated circuit that include multiple components on a single substrate are specifically configured to conserve power usage (i.e. in order to use the power source more efficiently). More specifically, in some embodiments, a single substrate of an application specific integrated circuit includes a transmitter and a receiver in order to conserve power usage with respect to the transmitter. These embodiments, are more specifically beneficial, when, for example, a transmitter as used herein is configured to transmit a relatively high-power driven signal such as, for example, a Bluetooth signal.

Embodiments of an application specific integrated circuit include dual mode radio transmitter and receivers as well as ultra-wide band (UWB) and narrow band communication capability to provide secure data transmission.

In some embodiments the incoming signals consist of different radio frequency bands including but not limited to 2.4 GHz 802.11b Wi-Fi transceiver, 2.36-2.4 GHz medical-hand transceiver, and 3.1-10 GHz 802.15.6 PHY-based UWB radio.

In some embodiments the physical parameter measuring device can be configured in different settings such as but not limited to home setting or hospital setting in which the application specific integrated circuit enables transmitting data through specific channels to ensure the data security. Described herein is an application specific integrated circuit configured for use with a physical parameter measuring device, said application specific integrated circuit comprising: a single substrate material to which are coupled: a cardiovascular monitoring sensor configured to sense a cardiac parameter data of an individual when contacted by the skin of the individual; a receiver configured to receive incoming data; a microprocessor configured to receive the cardiac parameter data from the cardiovascular monitoring sensor and the incoming data from the receiver; a transmitter configured to transmit at least one of the cardiac parameter data and incoming data and other sensors configured to gather other information such as by not limited to measuring acceleration, PT-INR, blood glucose, stroke volume, respiration and air flow volume, body tissue state, bone state, pressure, physical movement, body fluid density, the individual's physical location or audible body sounds, or a combination thereof. In some embodiments, the physical parameter measuring device comprises a handheld ECG monitoring device and the integrated circuit is a component of the handheld ECG monitoring device. In some embodiments, the cardiac monitoring device comprises a wearable ECG monitoring device and the integrated circuit is a component of the wearable ECG monitoring device. In some embodiments, the application specific integrated circuit is configured to communicate with the cardiac monitoring device. In some embodiments, the substrate comprises a semiconductor material. In some embodiments, the sensor comprises an ECG electrode. In some embodiments, the sensor comprises a PPG sensor. In some embodiments, the sensor comprises an impedance sensor. In some embodiments, the incoming data comprises an instruction executable by the microprocessor configured to cause the microprocessor to activate the sensor. In some embodiments, the incoming data comprises an instruction executable by the microprocessor configured to cause the microprocessor to transmit the cardiac monitoring data to a computing device using the transmitter. In some embodiments, the incoming data comprises an instruction executable by the microprocessor configured to cause the microprocessor to analyze the cardiac monitoring data.

In some embodiments the analysis of physiological data is achieved by utilizing a machine-learning and artificial intelligence software module to identify abnormalities in the individual's physiological conditions. In some embodiments, a machine learning software module is trained using real-time physiological parameter recordings and data relating to the individual including their medical health records and physiological parameters related to response to a series of designed activities. Abnormalities in physiological signals include the presence or absence of an abnormality in the physiological parameters, and said abnormalities are identified along with any known resulting or associated disease, disorder or condition. Data relating to the individual includes but not limited to demographic data, medical history, clinical data (e.g. from a health record, including an Electronic Health Record), public health research database or encoded data. In some embodiments, the transmitter comprises a Bluetooth transmitter. In some embodiments, the transmitter comprises a Wi-Fi transmitter. In some embodiments the transmitter is capable of ultra-wide band (UWB) and narrow band communication.

Described herein is an ECG monitoring device comprising an electrode configured to sense an electric potential on a first skin surface of a subject; a wireless transmitter configured to transmit a first wireless signal to a computing device; a receiver configured to receive a second wireless signal from the computing device; a microprocessor coupled to the first electrode, the wireless transmitter, and the receiver; and a non-transitory computer readable storage medium encoded with a computer program including instructions executable by the microprocessor that cause the microprocessor to: transmit the wireless signal with the wireless transmitter to the computing device, and execute an instruction associated with the second wireless signal that is received by the receiver from the computing device. In some embodiments, the first wireless signal comprises an instruction executable by the microprocessor configured to cause the microprocessor to activate the electrode. In some embodiments, the incoming data comprises an instruction executable by the microprocessor configured to cause the microprocessor to transmit the second wireless signal to the computing device using the transmitter. In some embodiments, the incoming data comprises an instruction executable by the microprocessor configured to cause the microprocessor to analyze the electrical potential. In some embodiments, the transmitter comprises a Bluetooth transmitter. In some embodiments, the transmitter comprises a WiFi transmitter. Described herein is a handheld ECG monitoring device comprising: a first electrode configured to sense a first electrical potential sensed from a skin surface of an individual; a second electrode configured to sense a second electrical potential sensed from a skin surface of an individual; an application specific integrated circuit comprising: a single substrate material to which are coupled: a receiver configured to receive incoming data; a microprocessor configured to receive the cardiac parameter data from the cardiovascular monitoring sensor and the incoming data from the receiver; and a transmitter configured to transmit at least one of the cardiac parameter data and incoming data. In some embodiments, the incoming data comprises an instruction executable by the microprocessor configured to cause the microprocessor to activate at least one of the first electrode and the second electrode. In some embodiments, the incoming data comprises an instruction executable by the microprocessor configured to cause the microprocessor to transmit the first and the second electrical potential to a computing device using the transmitter. In some embodiments, the incoming data comprises an instruction executable by the microprocessor configured to cause the microprocessor to analyze the first and the second electrical potential. In some embodiments, the transmitter comprises a Bluetooth transmitter. In some embodiments, the transmitter comprises a Wi-Fi transmitter.

Described herein is a method for cardiovascular monitoring comprising: receiving a device comprising a sensor, a microprocessor, a receiver, and a transmitter; receiving a first signal with said receiver comprising an instruction; carrying out said instruction using said microprocessor; and sensing a cardiovascular parameter; and transmitting the cardiovascular parameter using the transmitter. In some embodiments, the sensor, the microprocessor, the receiver, and the transmitter are positioned on a single substrate. In some embodiments, the instruction causes the microprocessor to activate the sensor. In some embodiments, the instruction causes the microprocessor to transmit the cardiovascular parameter using the transmitter.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an exemplary embodiment of a hand-held device configured for measuring physical parameters.

FIG. 2 shows an exemplary embodiment of a wearable sensing device configured to measure physical parameters being worn on a chest of an individual.

FIG. 3 shows an exemplary embodiment of a wearable sensing device 300 configured for measuring physical parameters being worn on a wrist of an individual and comprising a smartwatch.

FIG. 4 shows a schematic representation of an exemplary embodiment of an application specific integrated circuit.

FIG. 5 shows an exemplary embodiment of a system comprising a wearable sensing device and a hand-held device.

FIG. 6 shows an exemplary embodiment of a system comprising a wearable sensing device worn on a chest of an individual and a wearable sensing device comprising a smartwatch.

FIG. 7 shows an exemplary embodiment of a system as described herein comprising a device such as a digital processing device.

FIG. 8 shows an exemplary embodiment of a method for measuring physical parameters.

FIG. 9 shows an exemplary embodiment of a method for live (real-time) measurement of physical parameters.

FIG. 10 shows an exemplary embodiment of a method for a stealth mode measurement of physical parameters.

FIG. 11 shows components that may be used to assemble an exemplary wearable device in the form of a wearable patch such as the wearable device of FIG. 2.

FIG. 12 shows an exemplary wearable in the form of a smartwatch.

FIG. 13 shows a schematic of an exemplary operative coupling of a wearable patch, on body sub-system 1300a (as in FIG. 11) and a wearable sub-system 1300 such as a smartwatch (as in FIG. 12).

FIG. 14 shows an exemplary embodiment of a device or system as described herein.

### DETAILED DESCRIPTION

Described herein are devices, systems, and methods for sensing a physical parameter of an individual. In some embodiments, a physical parameter of an individual comprises an ECG sensed from the individual. In some embodiments, a physical parameter comprises a heart rate of an individual. In some embodiments, a physical parameter comprises a blood pressure of an individual. Non-limiting examples of physical parameters sensed by embodiments of the devices, systems, and methods described herein include temperature, spO2, perspiration, EEG, and urine output. One having skill in the art will understand that many other physical parameters may be sensed with an appropriate sensor type that is integrated with or into the devices, systems, and methods described herein.

Generally, devices, systems, and methods described herein for physical parameter measurement include an application specific integrated circuit configured to facilitate effective physical parameter monitoring. For example, in some embodiments, an application specific integrated circuit as described herein is configured to use power efficiently in the device, system, or method that includes an application specific integrated circuit.

### Devices Configured for Physical Parameter Monitoring

A device as described herein is configured to sense a physical parameter of an individual. In some embodiments, a device as described herein is configured to sense multiple cardiovascular parameters. Non-limiting examples of cardiovascular parameters sensed by embodiments of the devices described herein include an ECG, a heart rate, motion, a blood pressure, a PPG, stroke volume, heart sound and an oxygen saturation level.

A device as described herein typically comprises: a housing and one or more sensors.

A housing for a device as described herein, in some embodiments, comprises a housing configured to be handheld by an individual and includes one or more sensors positioned on one or more surfaces of the device.

In some embodiments, a housing is further configured to integrate with a display and/or a user interface. A housing, in some embodiments is configured to be worn by an individual such as a smartwatch band or a wristlet. Similarly, a housing may comprise in some embodiments, any part of a smartwatch including a smartwatch housing that houses the display and computing components of a smartwatch.

In some embodiments, a housing is configured to either physically integrate with one or more sensors and/or provide a mechanical coupling mechanism that allows one or more sensors to detachably couple with the housing.

A housing is generally configured to contain within it one or more electronic components and, in some embodiments, at least some of said electronic components are positioned on an application specific integrated circuit.

In some embodiments, one or more ECG sensors are positioned on a surface of a housing. For example, in some embodiments, a device comprises a hand-held housing and two ECG sensing electrodes are integrated with the housing and positioned on the same surface of the housing. In some embodiments, a housing comprises a wearable wristlet with a first ECG sensor positioned on a first surface of the wristlet (continuously in contact with a skin surface of an individual when worn) and a second ECG sensor positioned on a second surface of the wristlet (facing away from the individual when worn).

In some embodiments, a housing comprises at least one ECG sensor and at least one PPG sensor. In some embodiments, a housing comprises at least one ECG sensor, a PPG sensor, and a blood pressure sensor. In some embodiments, a housing comprises at least one ECG sensor, a PPG sensor, and a blood pressure sensor. In some embodiments, a housing comprises at least one ECG sensor, a PPG and an oxygen saturation level sensor. In some embodiments, a housing comprises at least one ECG sensor, a PPG, a blood pressure sensor and an oxygen saturation level sensor. In some embodiments, a housing comprises at least one ECG, and a blood pressure sensor. In some embodiments, a housing comprises at least one ECG, and an oxygen saturation level sensor. In some embodiments, a housing comprises at least one ECG, a blood pressure sensor and an oxygen saturation level sensor. In some embodiments, a housing comprises at least one PPG sensor and a blood pressure sensor. In some embodiments, a housing comprises at least one PPG sensor and an oxygen saturation level sensor. In some embodiments, a housing comprises at least one PPG sensor, a blood pressure sensor and an oxygen saturation level sensor. In some embodiments, a housing comprises a heart rate sensor and a blood pressure sensor. In some embodiments, a housing comprises a heart rate sensor, a blood pressure sensor and an oxygen saturation level sensor. In some embodiments, a wristlet housing comprises multiple PPG sensors separated precisely at fixed distances along the axis of vascular wall. In some embodiments, multiple sensors can be distributed over both housings and operate cooperatively. In some embodiments, at least one ECG sensor and one PPG sensor are allocated on a wristlet housing and an oxygen saturation sensor is located on the handheld housing. In some embodiments, a wristlet housing comprises a heart rate sensor and a blood pressure sensor while a handheld housing comprises an oxygen saturation sensor. In some embodiments, a wristlet housing comprises at least one ECG, a PPG and a blood pressure sensor, while the handheld housing comprises at least one ECG and an oxygen saturation level sensor. In some embodiments, a wristlet housing comprises at least one ECG and a blood pressure sensor, while the handheld housing comprises at least one ECG and an oxygen saturation level sensor. In some embodiments, a wristlet housing comprises at least one PPG and a blood pressure sensor, while the handheld housing comprises at least one ECG and an oxygen saturation level sensor.

In some embodiments, a housing also contains an application specific integrated circuit within it. In some embodiments, an application specific integrated circuit is a separate component that communicates with the device described herein but is not integrated with, coupled to, or contained within the housing.

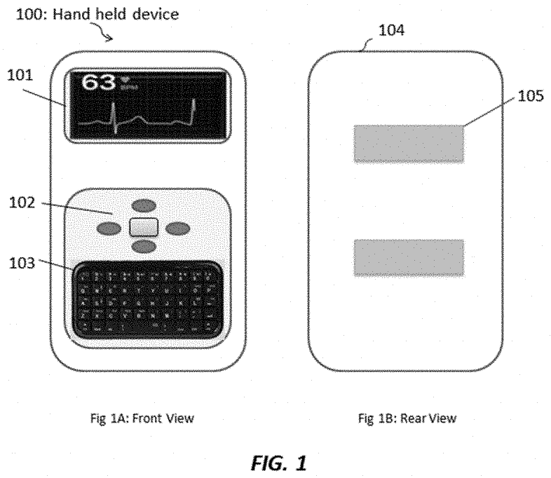

FIG. 1 shows an exemplary embodiment of a hand-held device 100 configured for physical parameter measurement. In this embodiment, the device comprises a display unit 101, a control navigation console 102, and an optional data entering keyboard 103 on the front of the device housing 104. Alternative embodiments, include a touch screen (not shown) which is either integrated with navigation console 102 or separate from navigation console 102. Two electrodes 105 are located on the back of the device housing 104, upon which one of each hand respectively touches one of two electrodes. The electrodes, in some embodiments, are spaced evenly, and, in some embodiments, are spaced unevenly.

With each electrode touching a separate hand, the sensing circuit is established. ECG data is then recorded in real-time and displayed on the display unit 101. Apart from real-time display of cardiovascular monitoring parameters, display unit 101 also serves as a user interface to display the control menu, functional selections, and/or interactive user environment to provide user feedback. In an exemplary embodiment, the device comprises two input interfaces: a five-control navigation console 102 and a data input keyboard 103 (or touch screen).

The five-control navigation console 102 provides simple gaming like navigation control through four arrow keys and one central joystick, while data input keyboard 103 provides data input including but not limited to notes, annotation, and user information. These data input interfaces 102 and 103 can be used separately or as a combination. In some embodiments, the data input interface comprises only one 5-control navigation console 102. In some embodiments, the data input interface comprises only keyboard 103. In some embodiments, the data input interface comprises both 102 and 103. In this embodiment 100, electrodes 105 are located at the back of the device. The placement of these electrodes varies with effective operation of the device. In some embodiments, electrodes are placed on the front of handheld device 100 and collocated on either sides of the 5-key navigation console 102, and monitoring is initiated by placing two thumbs of the user on the electrodes. In some embodiments, the electrodes can be integrated directly with keyboard 103 as two function keys located at sides of the keyboard 103 and/or as one of two arrow keys on navigation console 102.

It should be understood that while hand-held device 100 is configured in the exemplary embodiment of FIG. 1 to sense an ECG of an individual, many other physical parameters may be sensed with an appropriate sensor type that is integrated with or otherwise communicates with the hand-held device 100. For example, in other embodiments, the hand-held device 100 may be further configured to sense other physical parameters of an individual in addition or alternative to sensing an ECG. For example, in an embodiment of a hand-held device as described herein, a hand-held device comprises two ECG sensing electrodes and a PPG sensor. In this exemplary embodiment, a PPG sensor may be a wearable sensor that is worn by an individual to provide a continuous PPG recording. A processor of hand-held device 100 may be configured in this embodiment to analyze the continuously sensed PPG recording in order to determine when a PPG recording occurs that indicates that an ECG should be sensed.

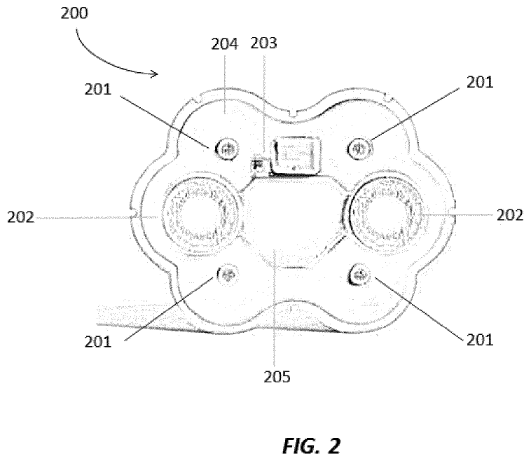

FIG. 2 shows an exemplary embodiment of a wearable sensing device 200 configured for measuring physical parameters shown being worn on a chest of an individual in the form of a patch shaped device. In this embodiment, a wearable sensing device comprises four primary thin film electrodes 201 configured for sensing an ECG of an individual wearing said wearable sensing device 200, two encased coin batteries 202, radio communication periphery 203, cardiovascular monitoring application specific integrated circuit (ASIC) 205.

All components 201, 202, 203, 205 are encased in a polymeric matrix. The casing 204 materials are selected for their biocompatibility, flexibility and high tearing strength. In some embodiments, material is PMMA interlaced with polyethylene as strong substrates for encased wiring. Many suitable materials can be used for the casing purpose as long as the biocompatibility and high tearing stress criteria can be satisfied. To maintain the comfort, the entire housing needs to be thin and conformed directly onto the human body. In some embodiments, two coin batteries 202 are evenly distributed symmetrical around the entire device 200 for maximum flexibility and wearing conformity. The batteries are encased within polymer matrix and connect to sensors 201, radio communication periphery 203 and ASIC 205. In some embodiments four circular electrodes 201 are symmetrically placed in the device 200 with wires completely embedded in the matrix. The electrodes 201 are partially embedded with partially raised metallic disk to ensure proper contact with the chest of an individual. Sensor data collected through electrodes will be transmitted via embedded wires to ASIC 205 for data analysis and storage. The storage data will then be transmitted via radio communication periphery 203 including but not limited to wireless, Bluetooth and RF communication to handheld device 100 or wristlet device 300.

Wearable sensing device 200 may be integrated into a garment worn by the individual so that the wearable sensing device 200 is positioned over the chest of the individual when worn (e.g. integrated within a shirt). In other embodiments, wearable sensing device 200 may be positioned on a chest of an individual using a strap or adhesive.

Wearable sensing device 200 may include 2 ECG sensing electrodes in some embodiments. Wearable sensing device 200 may include 3 ECG sensing electrodes in some embodiments. Wearable sensing device 200 may include 4 ECG sensing electrodes in some embodiments. Wearable sensing device 200 may include 5 ECG sensing electrodes in some embodiments. Wearable sensing device 200 may include 6 ECG sensing electrodes in some embodiments. Wearable sensing device 200 may include 7 ECG sensing electrodes in some embodiments. Wearable sensing device 200 may include 8 ECG sensing electrodes in some embodiments. Wearable sensing device 200 may include 9 ECG sensing electrodes in some embodiments. Wearable sensing device 200 may include 10 ECG sensing electrodes in some embodiments. In some embodiments, one or more ECG sensing electrodes are integrated directly into the wearable sensing device 200 and in some embodiments, one or more ECG sensing electrodes operatively coupled with the wearable sensing device 200 either through a wired or wireless connection.

In some embodiments, a wearable device 200 includes enough ECG sensing electrodes (either directly integrated or operatively coupled with the device) to record an ECG that provides clinical utility in a hospital setting such as a cardiac step-down unit or ICU. For example, in some embodiments, a wearable device 200 is configured to sense a 6 lead ECG. In some embodiments, the 6 lead ECG comprises all six precordial leads sensed by six electrodes positioned on the surface of the wearable device 200 so that they respectively sense the 6 precordial leads when placed over the chest of the individual. For example, in some embodiments, a wearable device 200 is configured to sense a 12 lead ECG.

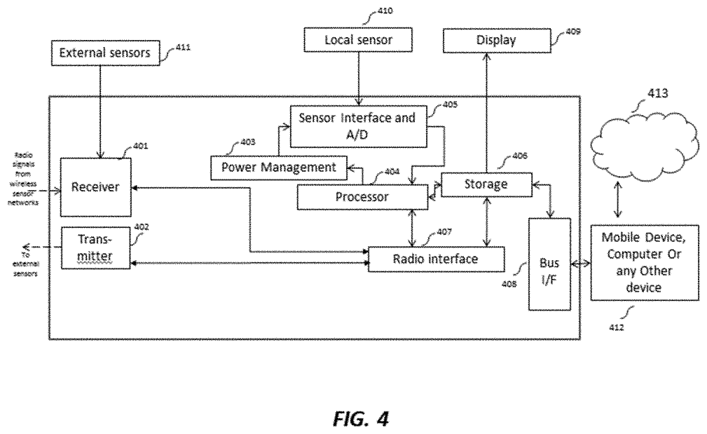

Wearable device 200 may be configured to operatively communicate with other sensors or wearable devices. For example, in some embodiments, an application specific integrated circuit (ASIC) as described in FIG. 4 below is integrated into the wearable device 200 and provides communication means between the wearable device 200 and other sensors and/or devices. For example, a smartwatch may be configured to sense a lead I ECG tracing and a wearable device 200 may be configured to sense precordial leads V1-V6, wherein the smartwatch is further configured to integrate with the precordial leads so that the wearable device 200 records a seven lead ECG. Likewise, two wristlets containing ECG sensing electrodes and one electrode configured to contact a lower extremity may integrate with a wearable device 200 that includes six electrodes so that all 12 leads are sensed when the wristlet electrodes and the lower extremity electrode communicate with the wearable device 200. Such communication is facilitated by an ASIC as described.

In some embodiments, wearable device 200 is configured to select among a set of radios for both incoming signals (multiple sensors) and outgoing signals (to the cloud) by sending out test packets and checking how much bandwidth and accuracy is attained by each mode of communications periodically. This dynamic method of gateway is advantageous in that it for example allows the wearable device 200 to adapt to changing bandwidth and allows new sensors to be added on-the-fly.

It should be understood that while wearable sensing device 200 is configured in the exemplary embodiment of FIG. 2 to sense an ECG of an individual, many other physical parameters may be sensed with an appropriate sensor type that is integrated with or otherwise communicates with the wearable sensing device 200. For example, in other embodiments, the wearable sensing device 200 may be further configured to sense other physical parameters of an individual in addition or alternative to sensing an ECG. For example, in an embodiment of a hand-held device as described herein, a hand-held device comprises two ECG sensing electrodes and a PPG sensor. In this exemplary embodiment, a PPG sensor may be a wearable sensor that is worn by an individual to provide a continuous PPG recording. A processor of wearable sensing device 200 may be configured in this embodiment to analyze the continuously sensed PPG recording in order to determine when a PPG recording occurs that indicates that an ECG should be sensed.

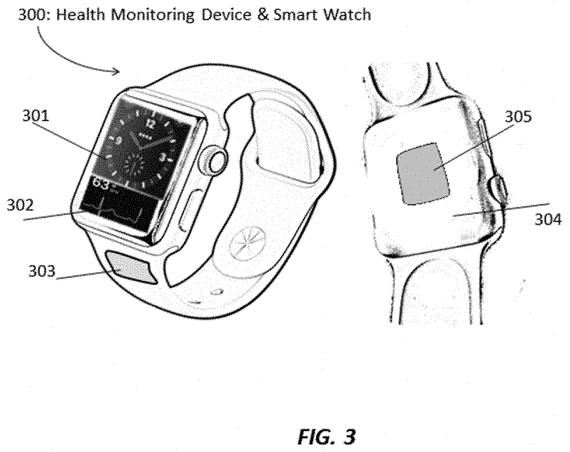

FIG. 3 shows an exemplary embodiment of a wearable sensing device 300 configured for measuring physical parameters shown being worn on a wrist of an individual and comprising a smartwatch. The wearable sensing device 300 integrates the functionality of a health monitoring device 100, data receiver and processor for an external monitoring device 200, and a smartwatch. The device comprises a touch screen display integrated over the front of a smart watch housing 304. In some embodiments the display provides time information 301 and cardiovascular monitoring parameters 302 including ECG, PPG, heart rate, blood pressure and oxygen saturation level. In some embodiments the screens 301 and 302 can be either displayed simultaneously or switchable by controls. In some embodiments, two sensor electrodes are placed strategically on the housing. One electrode 305 is placed on the back side of the watch housing 304 that makes constant contact with the skin surface of an individual. The other electrode 303 is embedded in the wrist band of the watch 300. In this embodiment, ECG monitoring process is activated by the individual who is wearing the watch 300 over one wrist placing one finger of the other hand over the electrode on the wrist band 303, until a stable reading is established and displayed on display 302. In some embodiments, the activation is also established by placing one finger from one hand on electrode 305 and placing one finger from the other hand on electrode 303. The collected data will be stored, analyzed in-situ and/or transmitted directly to external server 412 or cloud storage 413.

It should be understood that while wearable sensing device 300 is configured in the exemplary embodiment of FIG. 3 to sense an ECG of an individual, many other physical parameters may be sensed with an appropriate sensor type that is integrated with or otherwise communicates with the wearable sensing device 300. For example, in other embodiments, the wearable sensing device 300 may be further configured to sense other physical parameters of an individual in addition or alternative to sensing an ECG. For example, in an embodiment of a hand-held device as described herein, a hand-held device comprises two ECG sensing electrodes and a PPG sensor. In this exemplary embodiment, a PPG sensor may be a wearable sensor that is worn by an individual to provide a continuous PPG recording. A processor of wearable sensing device 300 may be configured in this embodiment to analyze the continuously sensed PPG recording in order to determine when a PPG recording occurs that indicates that an ECG should be sensed.

FIG. 4 shows a schematic representation of an exemplary embodiment of an application specific integrated circuit 400. In this embodiment, the application specific integrated circuit (ASIC) 400 comprises central processor 404, sensor interface and A/D module 405, on-chip power management unit 403, on-chip data storage 406, and communication peripheries including but not limited to receiver 401, transmitter 402, radio interface 407, I/F bus 408. The ASIC 400 performs (i) integrated data collections from variety of directly connected sensors including but not limited to wired or wireless external sensors 411, locally integrated sensors 410 as well as the wirelessly connected sensor networks; (ii) real-time data processing including but not limited to data encryption/decryption, data compression and decompression, real-time data analysis to extract cardiovascular parameters including but not limited to heart rate, blood flow rate, blood pressure, oxygen saturation level, skin conductivity; (iii) data management and communication that allows both on-chip data storage 406 and communication with remotely connected computing devices 412 and cloud computing server 413. The power consumption is regulated by an on-chip power management unit 403 to optimize performance and minimize power consumption. In some embodiments the ASIC reconfigures its functionality based on available sensors and sensor networks as well as the available computation resources. In some embodiments, when ASIC is interfaced with local sensor 410 and at least one mobile device, the sensor data will be collected through on-chip sensor interface and A/D unit, processed by on-chip processor, cached by on-chip storage, and finally transmitted to a mobile computing device 412 via I/F Bus 408 and subsequently cloud server 413 for further analysis and data aggregation. The mobile computing devices include but not limited to mobile devices, smart phones, and computer servers. In some embodiments, when ASIC is interfaced with external sensors 411 and at least one mobile computing device, the cardiovascular signal is collected off-chip by the external sensors and transmitted to ASIC via Receiver 401 and Radio interface 407. The signal will be processed by an on-chip processor 404 and cached to on-chip storage 406, subsequently transmitted to a mobile computing device 412 via I/F Bus 408 for further analysis and data aggregation. Device control signal is transmitted via Radio interface to transmitter 402. External sensors include but not limited to single wirelessly connected sensors or multiple linked wireless sensors forming a sensor network. In some embodiments, when ASIC is interfaced with external sensor 411 and no mobile device, the cardiovascular signal is collected off-chip by the external sensors and transmitted to ASIC via Receiver 401 and Radio interface 407. The signal is processed by an on-chip processor 404 and stored in on-chip storage 406, and downloaded later to a mobile computing device 412 via I/F Bus 408 when connection is established.

### Systems Configured for Physical Parameter Measurement

A system as described herein is generally configured to monitor a physical parameter of an individual and comprises a device as described herein. In some embodiments, a system as described herein comprises a network element for communicating with a server. In some embodiments, a system as described herein comprises a server. In some embodiments, the system is configured to upload to and/or download data from the server. In some embodiments, the server is configured to store sensor data, and/or other information for the individual. In some embodiments, the server is configured to store historical data (e.g., past sensor data).

In some embodiments, a device as described herein comprises a digital processing device that includes one or more hardware central processing units (CPUs) or general purpose graphics processing units (GPGPUs) that carry out the device's functions. The digital processing device further comprises an operating system configured to perform executable instructions. The digital processing device is optionally connected to a computer network. The digital processing device is optionally connected to the Internet such that it accesses the World Wide Web. The digital processing device is optionally connected to a cloud computing infrastructure. Suitable digital processing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will recognize that many smartphones are suitable for use in the system described herein.

Typically, a digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing.

A digital processing device as described herein either includes or is operatively coupled to a storage and/or memory device. The storage and/or memory device is one or more physical devices used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

Some embodiments of the systems described herein are computer based systems. These embodiments include a CPU including a processor and memory which may be in the form of a non-transitory computer readable storage medium. These system embodiments further include software that is typically stored in memory (such as in the form of a non-transitory computer readable storage medium) where the software is configured to cause the processor to carry out a function. Software embodiments incorporated into the systems described herein contain one or more modules.

Some of the software embodiments described herein are configured to cause a processor to receive data from a sensor. Some of the software embodiments described herein are configured to cause a processor to transmit data from a sensor. Some of the software embodiments described herein are configured to cause a processor to analyze data from a sensor

In various embodiments, a device comprises a computing device or component such as a digital processing device. In some of the embodiments described herein, a digital processing device includes a display to send visual information to a user. Non-limiting examples of displays suitable for use with the systems and methods described herein include a liquid crystal display (LCD), a thin film transistor liquid crystal display (TFT-LCD), an organic light emitting diode (OLED) display, an OLED display, an active-matrix OLED (AMOLED) display, or a plasma display.

A digital processing device, in some of the embodiments described herein includes an input device to receive information from a user. Non-limiting examples of input devices suitable for use with the systems and methods described herein include a keyboard, a mouse, trackball, track pad, or stylus. In some embodiments, the input device is a touch screen or a multi-touch screen.

The systems and methods described herein typically include one or more non-transitory computer readable storage media encoded with a program including instructions executable by the operating system of an optionally networked digital processing device. In some embodiments of the systems and methods described herein, the non-transitory storage medium is a component of a digital processing device that is a component of a system or is utilized in a method. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

Typically the systems and methods described herein include at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages. The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

Typically, the systems and methods described herein include and/or utilize one or more databases. In view of the disclosure provided herein, those of skill in the art will recognize that many databases are suitable for storage and retrieval of baseline datasets, files, file systems, objects, systems of objects, as well as data structures and other types of information described herein. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In other embodiments, a database is based on one or more local computer storage devices.

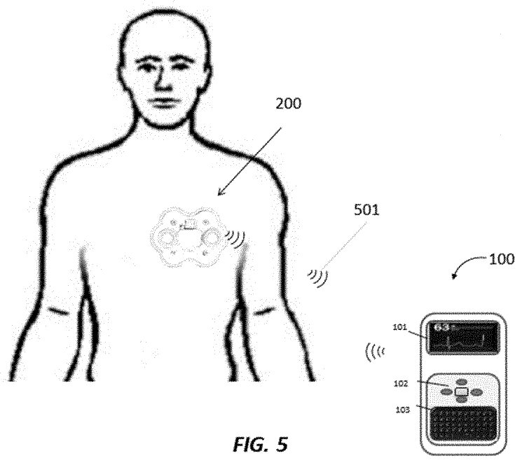

FIG. 5 shows an exemplary embodiment of a system comprising a wearable sensing device and a handheld device. In this embodiment, the handheld device 100 is in wireless communication 501 with the external sensor 200. Data and instructions can be communicated between the handheld device 100 and the external sensor via but not limited to Wi-Fi or Bluetooth communication. In some embodiments the external sensor is a disposable wearable patch worn on a chest of an individual, monitoring cardiovascular function. In some embodiments the handheld device 100 functions as an event monitor and the external sensor 200 acts as a long term Holter monitor, continuously monitoring the activities of heart for long periods of time. In this embodiment the cardiovascular monitoring data as well as any other sensor data can be either constantly or periodically or on a timely based manner transmitted to and recorded at the handheld device 100. The sensor data can be stored in a local memory of the handheld device. The recorded data can then be transmitted to any other computing device or to a remote cloud computing server. The cardiovascular data is transmitted via radio communication periphery of the external sensor via but not limited to wireless, Bluetooth and RF communication to handheld device 100.

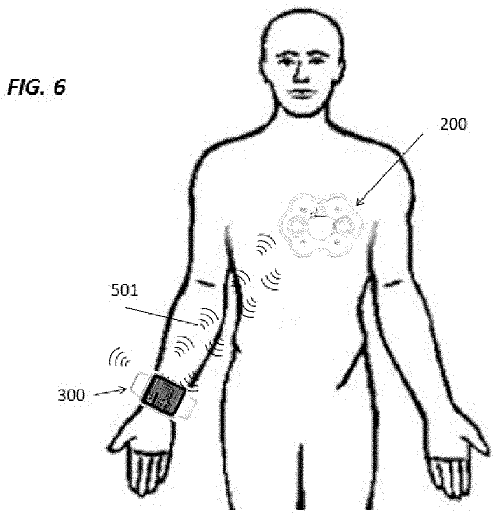

FIG. 6 shows an exemplary embodiment of a system comprising a wearable sensing device worn on a chest of an individual and a wearable sensing device comprising a smartwatch. In this embodiment, the wristlet device 300 is in wireless communication 501 with the external sensor 200. Data and instructions can be communicated between the wristlet device 300 and the external sensor via but not limited to Wi-Fi or Bluetooth communication. In some embodiments the external sensor is a disposable wearable patch worn on a chest of an individual, monitoring cardiovascular function. In some embodiments the wristlet device 300 functions as an event monitor and the external sensor 200 acts as a long term Holter monitor, continuously monitoring the activities of heart for long periods of time. In this embodiment the cardiovascular monitoring data as well as any other sensor data can be either constantly or periodically or on a timely based manner transmitted to and recorded at the wristlet device 300. The sensor data can be stored in a local memory of the wristlet device. The recorded data can then be transmitted to any other computing device or to a remote cloud computing server. The cardiovascular data is transmitted via radio communication periphery of the external sensor via but not limited to wireless, Bluetooth and RF communication to wristlet device 300.

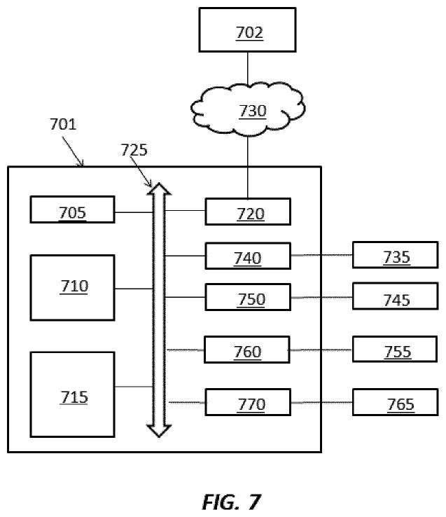

FIG. 7 shows an exemplary embodiment of a system as described herein comprising a device such as a digital processing device 701. The digital processing device 701 includes a software application configured to monitor the physical parameters of an individual. The digital processing device 701 may include a central processing unit (CPU, also “processor” and “computer processor” herein) 705, which can be a single core or multi-core processor, or a plurality of processors for parallel processing. The digital processing device 701 also includes either memory or a memory location 710 (e.g., random-access memory, read-only memory, flash memory), electronic storage unit 715 (e.g., hard disk), communication interface 720 (e.g., network adapter, network interface) for communicating with one or more other systems, and peripheral devices, such as cache. The peripheral devices can include storage device(s) or storage medium 765 which communicate with the rest of the device via a storage interface 770. The memory 710, storage unit 715, interface 720 and peripheral devices are configured to communicate with the CPU 705 through a communication bus 725, such as a motherboard. The digital processing device 701 can be operatively coupled to a computer network (“network”) 730 with the aid of the communication interface 720. The network 730 can comprise the Internet. The network 730 can be a telecommunication and/or data network.

The digital processing device 701 includes input device(s) 745 to receive information from a user, the input device(s) in communication with other elements of the device via an input interface 750. The digital processing device 701 can include output device(s) 755 that communicates to other elements of the device via an output interface 760.

The CPU 705 is configured to execute machine-readable instructions embodied in a software application or module. The instructions may be stored in a memory location, such as the memory 710. The memory 710 may include various components (e.g., machine readable media) including, but not limited to, a random access memory component (e.g., RAM) (e.g., a static RAM “SRAM”, a dynamic RAM “DRAM, etc.), or a read-only component (e.g., ROM). The memory 710 can also include a basic input/output system (BIOS), including basic routines that help to transfer information between elements within the digital processing device, such as during device start-up, may be stored in the memory 710.

The storage unit 715 can be configured to store files, such as health or risk parameter data, e.g., individual health or risk parameter values, health or risk parameter value maps, and value groups. The storage unit 715 can also be used to store operating system, application programs, and the like. Optionally, storage unit 715 may be removably interfaced with the digital processing device (e.g., via an external port connector (not shown)) and/or via a storage unit interface. Software may reside, completely or partially, within a computer-readable storage medium within or outside of the storage unit 715. In another example, software may reside, completely or partially, within processor(s) 705.

Information and data can be displayed to a user through a display 735. The display is connected to the bus 725 via an interface 740, and transport of data between the display other elements of the device 701 can be controlled via the interface 740.

Methods as described herein can be implemented by way of machine (e.g., computer processor) executable code stored on an electronic storage location of the digital processing device 701, such as, for example, on the memory 710 or electronic storage unit 715. The machine executable or machine readable code can be provided in the form of a software application or software module. During use, the code can be executed by the processor 705. In some cases, the code can be retrieved from the storage unit 715 and stored on the memory 710 for ready access by the processor 705. In some situations, the electronic storage unit 715 can be precluded, and machine-executable instructions are stored on memory 710.

In some embodiments, a remote device 702 is configured to communicate with the digital processing device 701, and may comprise any mobile computing device, non-limiting examples of which include a tablet computer, laptop computer, smartphone, or smartwatch. For example, in some embodiments, the remote device 702 is a smartphone of the user that is configured to receive information from the digital processing device 701 of the device or system described herein in which the information can include a summary, sensor data, or other data. In some embodiments, the remote device 702 is a server on the network configured to send and/or receive data from the device or system described herein.

### Methods for Physical Parameter Monitoring

Described herein are methods for physical parameter monitoring. Generally, a method as described herein includes use of any of the devices and/or systems described herein in monitoring one or more physical parameters of an individual.

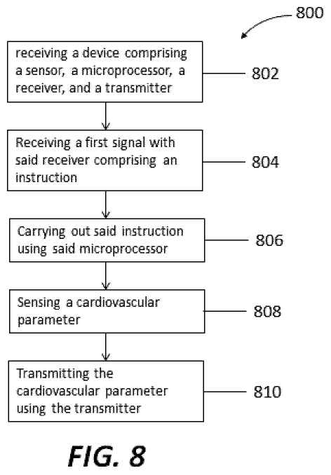

FIG. 8 shows an exemplary embodiment of a method 800 for physical parameter measurement. In a step 802 a device comprising a sensor, a microprocessor, a receiver, and a transmitter is received by an individual. In some embodiments after device initialization, the device performs automatic fault check, battery status check, sensor calibration and establishes communication with control unit of the processor 404. In some embodiments after the device is properly initialized, device remains in the idle mode to conserve battery power. In idle mode, the device scans communication ports for instruction. In a step 804, a first signal comprising an instruction is received by the receiver of the device to activate the monitoring function. An acknowledgement signal will be transmitted back to control unit of the processor 404 to establish a synchronized mode. The device then wakes up from idle mode to active acquisition mode to perform an instruction. The signal may comprise a single instruction or a set of instructions. In some embodiments, the signal comprises a single query that instructs device to perform a single instance physical parameter measurement and transmit back to control unit of the processor 404 including but not limited to heart rate, blood flow rate, blood pressure, and oxygen saturation level. In a step 806, the microprocessor carries out the single instance measurement by activating the sensor 303, collecting the data and compiling data into single data packet containing abovementioned physical parameters. In a step 808, the transmitter transmits this data packet containing said single instance physical parameter. In some embodiments after the task is complete, the device goes back to idle mode. In some embodiments, the signal comprises a set of instructions to perform a burst of measurements during a specified period of time. For example, the signal comprises the instruction to acquire a bust of 10 measurements at an interval of 1 minute. In a step 806, the microprocessor performs 10 said measurements, and packages the entire data into one data packet or multi packets at the end of measurement. In a step 808, the transmitter sends the data packet back to control unit of the processor 404 or data aggregator 412. In some embodiments after the task completion, the device goes into idle mode.

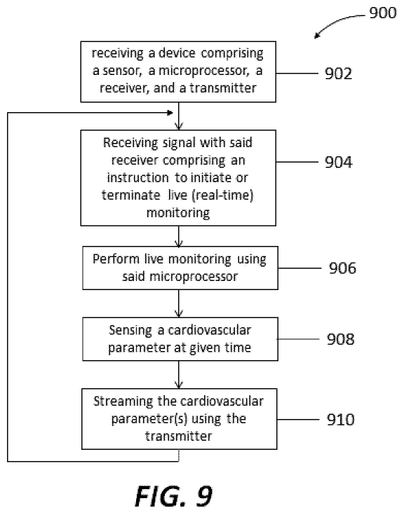

FIG. 9 shows an exemplary embodiment of a method 900 for live (real-time) physical parameter monitoring. In a step 902 a wearable device comprising a sensor, a microprocessor, a receiver, and a transmitter is received by an individual. In some embodiments after device initialization, the device performs automatic fault check, battery status check, sensor calibration and establishes communication with control unit of the processor 404. After the device is properly initialized, device remains in the idle mode to conserve battery power. In a step 904, a signal comprising an instruction to either initiate or terminate a live (real-time) monitoring mode is received by the receiver of the device. An acknowledgement signal will be transmitted back to control unit of the processor 404 to establish live (real-time) mode link. In some embodiments the device will wake up from idle mode to real-time mode. In a step 906, the microprocessor 404 initiates an endless timer that carries out sensor measurements periodically or on a timely basis. The acquired physical parameters at any given time are packaged individually and form a series of real-time data stream. In a step 908, the transmitter transmits the real-time data stream directly to control unit of the processor 404 and/or data aggregators 412. In some embodiments if a termination signal for the real-time mode is received in step 904, the data stream is stopped and the device goes into idle.

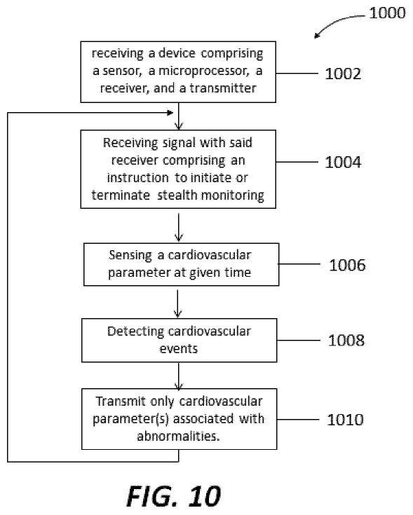

FIG. 10 shows an exemplary embodiment of a method 1000 for a stealth mode physical parameter monitoring. In a step 1002 a wearable device comprising a sensor, a microprocessor, a receiver, and a transmitter is received by an individual. In some embodiments after device initialization, the device performs automatic fault check, battery status check, sensor calibration and establishes communication with control unit of the processor 404. In some embodiments after the device is properly initialized, device requests a series of tests to establish the normal physical conditions including but not limited to quiet siting and workout. These nominal conditions can be established through static, dynamic and heuristic regression using artificial intelligence. In some embodiments, the nominal conditions are pre-compiled and uploaded by control unit of the processor 404 or data aggregators 412. In some embodiments, the nominal conditions are established through several routine activities. In some embodiments, the nominal conditions are constructed through on-chip data analysis of real-time stream recording. In a step 1004, a signal comprising an instruction to either initiate or terminate a stealth monitoring mode is received by the receiver of the device. In some embodiments an acknowledgement signal will be transmitted back to control unit of the processor 404 to establish communication link. In some embodiments after an initiation signal is received, the device will wake up from idle mode to real-time mode. In a step 1006, sensors acquire said physical parameters continuously. In a step 1008, the real-time data stream comprising instantaneous physical parameters are compared with the established nominal condition. If an abnormal event is detected, the segment of data streamer prior to and immediately after the event will be packaged by processor 404. In a step 1010, if an abnormal event is detected, the packaged segment of data stream is transmitted by the transmitter to control unit of the processor 404 and/or data aggregators 412. In some embodiments if a termination signal for stealth mode is received in step 1004, the device goes into idle mode.

An exemplary embodiment of method for providing individual based physical parameter monitoring using artificial intelligence is provided here. In this embodiment, the device provides individual based monitoring by constructing individual based baseline of one's physical conditions including but not limited to heart rate, oxygen saturation level, stroke volume and blood pressure and detecting cardiovascular anomalies in real-time using artificial intelligence. To construct one's baseline cardiovascular information, the wearable device comprising a sensor, a microprocessor, a receiver, and a transmitter will, via user input, collects user specific information including but not limited to age, gender, weight, race, and height. User specific information combined with available medical studies results and individual's medical health records will enter into an artificial intelligence (AI) data aggregator to determine the normal range of physiological signals for given race, body mass index, and gender. In some embodiments, the artificial intelligence data aggregators may consist of multi-layer neural networks. In some embodiments, AI aggregators may consist of a supported vector machine (SVM) multi-variant classifier. In some embodiments, AI aggregators may consist of a fuzzy logic multi-variant Gaussian classifier. Additional user specific baseline conditions will be further established after the individual completes a series of specific activity tasks including but not limited to sleeping, sitting quietly, exercising, and walking. The physiological parameters are recorded and ensemble averaged based on each activity type to train on-board AI or machine learning software to establish activity-related baseline conditions. Real-time recorded physiological signals are compared with these activity-related baseline conditions using on-board AI software to detect anomalies. Once the anomalies are detected, the events and related physiological signals are transmitted to external data aggregators or emergency responders.

An exemplary embodiment of method for providing smart behavior-based physiological monitoring using artificial intelligence is provided herein. In this embodiment, the wearable device provides physiological monitoring based on individual's behavior and activities as well as detection of anomalies. In this embodiment, the real-time physiological parameters including but not limited to heart rate, oxygen saturation level, stroke volume and blood pressure will be collected continuously by the wearable device comprising a sensor, a microprocessor, a receiver, a transmitter and other auxiliary sensors, and aggregated into different user behavior and activities by an on-board AI or machine learning software. In this embodiment, AI or machine learning software is trained by incorporating additional auxiliary sensors enabling behavioral analysis. These auxiliary sensors include but not limited to acceleration, skin conductivity, navigation sensors and clock. In some embodiments, low acceleration reading at night for extended period of time will be recognized by AI software as user in sleep state. The physiological signals will be classified as sleep state parameters. In some embodiments, high accelerometer reading and high heart rate may be recognized by AI software as user in the state of exercise. Hence physiological signals will be aggregated and compared as exercise-state parameters. In some embodiments, high accelerometer reading and low rate of change in locations may be recognized by AI software as user in the state of walking. Hence physiological signals will be aggregated and compared as walking-state parameters. In some embodiments, low accelerometer reading and high rate of change in locations may be recognized by AI software as user in the state of driving. Physiological signals will be aggregated and compared as driving-state parameters. In some embodiments, high accelerometer reading and large skin conductivity may be recognized by AI software as user in the state of excitement. Hence Physiological signals will be aggregated and compared as excitement-sate parameters. These smartly classified/aggregated Physiological parameters may be used to train AI or machine learning software to establish real-time behavior-based baseline database, with which the real-time signals are compared and subsequently anomalies are detected.

### Software for Physical Parameter Measurement

Described herein is software and software modules configured to enable the abovementioned methods for physical parameter monitoring. A system as described herein utilizes software as a component of one or more computing devices within the system, including a device as described herein.

In some embodiments, software is configured to enable the method of client server mode physical parameter monitoring. In this embodiment, software places the receiver in active listening mode and waits for a trigger signal. As a query signal comprising single or multiple measurement requests are received by the receiver, software executes a sequential task including activating the sensor, performing data acquisition of physical parameters, packaging the data, activating transmitter to transfer the data to client, and returning receiver back to active listening mode and putting the rest of device to idle.

In some embodiments, software is configured to enable the method of real-time physical parameter monitoring. In this embodiment, software places the receiver in a concurrent background listening mode and waits for an incoming signal that comprises either an initiation or a termination instruction. Software will also place the transmitter in a concurrent background routine with a first-in-first-out (FIFO) cache. The background routine of transmitter will actively scan the FIFO cache. When data packets are added into FIFO cache, the background routine will remove the first available data packet from FIFO and transmit it to control unit of processor 404 and data aggregators 412. The live (real-time) mode software is also arranged as a concurrent foreground routine, which is constantly scanning receiver cache. When an initiation instruction is received, the foreground main routine activates a software timer and starts the data acquisition at a short interval. After each acquisition, the software will package each measurement into a single data packet and insert into transmitter FIFO for real time streaming. When a termination instruction is received, the foreground main routine will terminate live data acquisition, stop the software timer and return to a state that software would be constantly waiting for another initiation instruction

In some embodiments, software is configured to enable the method of stealth mode physical parameter monitoring. In this embodiment, software places the receiver in a concurrent background listening mode and waits for an incoming signal that comprises either initiation or termination instruction of stealth mode. Software will also place the transmitter in a concurrent background routine with a first-in-first-out (FIFO) cache. The background routine transmitter will actively scan the FIFO cache. When data packets are added into FIFO cache, the background routine will remove the first available data packet from FIFO and transmit it to control unit of the processor 404 and data aggregators 412. The transmitting will continue until the FIFO is empty. In some embodiments the stealth mode software is also arranged as a concurrent foreground routine, which is constantly scanning receiver. When an initiation instruction is received, the foreground main routine activates a software timer and starts the data acquisition at a short interval. After each acquisition, the software will compare the instantaneous data to establish a nominal matrix. If the instantaneous data is beyond the nominal matrices, software will package a segment of measurements including time prior to and immediately after the current data into a single data packet and insert into transmitter FIFO for transmission. In some embodiments when a termination instruction is received, the foreground main routine will terminate stealth data acquisition, stop the software timer and return to a state of constantly waiting for another initiation instruction.

### Exemplary Embodiments

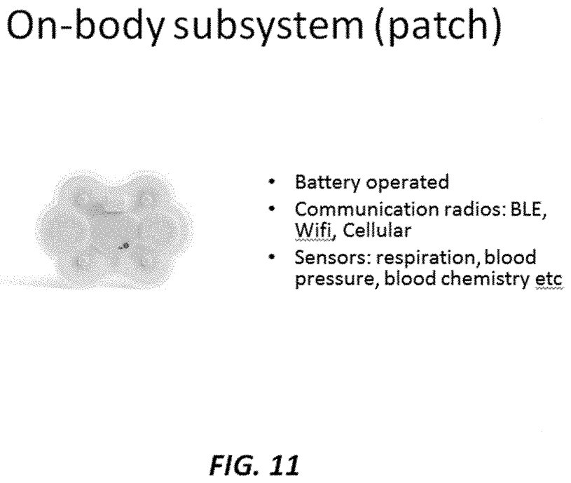

FIG. 11 shows an exemplary wearable device in the form of a wearable patch such as the wearable device of FIG. 2. The wearable patch of the example is powered by battery comprising, for example, a thin profile lithium battery. In a first option, ECG circuitry comprises, for example, an Analog Device AD8232/AD8233 circuit board. In a second option, ECG circuitry comprises, for example, a TI ADS1293 circuit board. Both ECG circuitries provide onboard circuitry for receiving and processing ECG signals received from one or more ECG electrodes. A CPU in the example shown comprises, for example, a Cypress PSoC-BLE which includes Bluetooth capability. A Nordic semi is utilized in an alternative embodiment. It should be understood that a wearable patch comprising a battery, ECG circuitry, a CPU, and that includes Bluetooth capability may be assembled to provide the functionality described herein in terms of, for example, sensing an ECG from one or more operatively coupled electrodes, communicating with other sensors and devices, and processing various sensed patient parameters including for example a PPG sensed by a PPG sensor that operatively couples with the wearable patch.

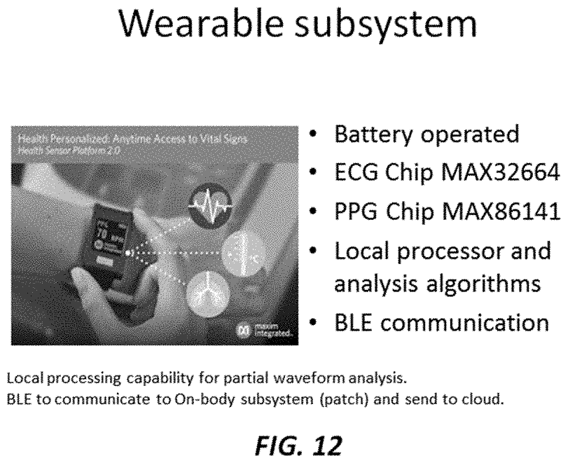

FIG. 12 shows an exemplary wearable in the form of a smartwatch. In this example, a smartwatch housing is 3D printed and includes both onboard PPG circuitry and ECG circuitry as well as Bluetooth capability. PPG circuitry may comprise, for example, products TI AFE4900 or AFE4499. It should be understood that a smartwatch including onboard PPG circuitry and ECG circuitry as well as Bluetooth capability may be assembled to provide the functionality described herein in terms of, for example, sensing an ECG from one or more operatively coupled electrodes, communicating with other sensors and devices, and processing various sensed patient parameters including for example a PPG sensed by a PPG sensor that operatively couples with the smartwatch.

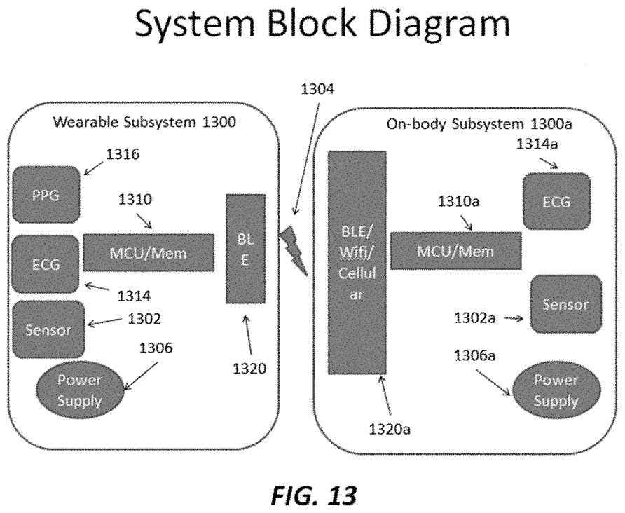

FIG. 13 shows a schematic of an exemplary operative coupling of a wearable patch, on body sub-system 1300a (as in FIG. 11) and a wearable sub-system 1300 such as a smartwatch (as in FIG. 12). A Bluetooth module 1320 is used to transmit and receive data from wearable sub-system 1300 to on-board sub-system 1300a through communication link 1304. In some embodiments, Bluetooth module 1320 is coupled to an antenna. In the embodiment shown, on body sub-system 1300a further includes additional communication capability such as a cellular connectivity capability for connecting to, for example, remote servers and/or the cloud. Also shown are multipoint control units (MCU) 1310 and 1310a which function as the central processor for the sub-systems 1300 and 1300a and carry out functions on each of the sub-systems 1300 and 1300a including: sending commands and collecting data for storage remotely and/or on onboard memories 1312 and 1312a, and fetching data from memories 1312 and 1312a to transmit to other devices, and/or sensors 1302 and 1302a, and/or remote servers and/or the cloud. The sub-systems 1300 and 1300a are both configured to accommodate different power sources 1306 and 1306a such as, for example, a lithium battery. Also included are ECG circuitries 1314 and 1314a as well as PPG circuitry 1316 are included in the circuitry embodiment shown.

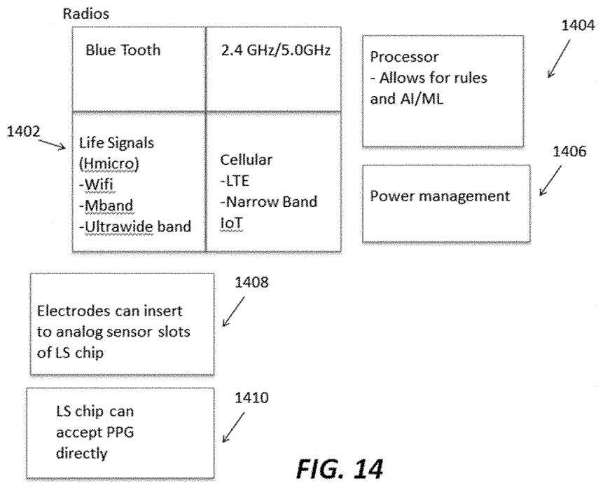

FIG. 14 shows an exemplary embodiment of a device or system as described herein. Radios 1410 may include one or more of Bluetooth, 2.4 GHz/5.0 GHz antenna, Wifi, Mband, and Ultrawide band modules, and Cellular, LTE and Narrow Band IoT. A processor 1404 which is configured to allow for rules and implements machine learning software. A power management module 1406 provides and controls power supply. Electrodes and other sensor types 1408 may either hard connect or wirelessly connect to the devices and systems described herein. In some embodiments, such sensors may also be accessed or otherwise communicate data with other devices and sensors. LS chip 1410 is configured to receive PPG data directly.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A physical parameter system comprising:
(A) an application specific integrated circuit comprising a single substrate material to which are coupled:
(i) a cardiovascular monitoring module configured to receive a sensed cardiac parameter data of an individual when a sensor operatively coupled to said cardiovascular monitoring module is contacted by the skin of the individual;
(ii) a receiver configured to receive incoming data;
(iii) a microprocessor configured to receive the cardiac parameter data from the cardiovascular monitoring module and the incoming data from the receiver; and
(iv) a transmitter configured to transmit at least one of the cardiac parameter data and incoming data.
(B) a physical parameter measuring device configured to operatively communicate with said application specific integrated circuit.

2. The system of claim 1, wherein the physical parameter measuring device comprises a handheld ECG monitoring device and the integrated circuit is a component of the handheld ECG monitoring device.

3. The system of claim 1, wherein the physical parameter measuring device comprises a wearable ECG monitoring device and the integrated circuit is a component of the wearable ECG monitoring device.

4. The system of claim 1, wherein the application specific integrated circuit is configured to communicate with a cardiac monitoring device that includes said sensor.

5. The system of claim 1, wherein the substrate comprises a semiconductor material.

6. The system of claim 1, wherein the sensor comprises an ECG electrode.

7. The system of claim 1, wherein the sensor comprises a PPG sensor.

8. The system of claim 1, wherein the sensor comprises an impedance sensor.

9. The system of claim 1, wherein the sensor is configured to measure at least one of acceleration, PT-INR, blood glucose, stoke volume, respiration and air flow volume, body tissue state, bone state, pressure, physical movement, body fluid density, the individual's physical location or audible body sounds, or a combination thereof.

10. The system of claim 1, wherein the incoming data comprises an instruction executable by the microprocessor configured to cause the microprocessor to activate the sensor.

11. The system of claim 1, wherein the incoming data comprises an instruction executable by the microprocessor configured to cause the microprocessor to transmit the cardiac monitoring data to a computing device using the transmitter.

12. The system of claim 1, wherein the transmitter is configured to communicate 3.1-10 GHz ultra-wide band (UWB), 2.4 GHz Wi-Fi, 2.36-2.4 GHz medical-band and narrow band radio signals.

13. The system of claim 1, wherein the incoming data comprises an instruction executable by the microprocessor configured to cause the microprocessor to analyze the cardiac monitoring data.

14. The system of claim 1, wherein the transmitter comprises a Bluetooth transmitter.

15. The system of claim 1, wherein the transmitter comprises a Wi-Fi transmitter.

16. An application specific integrated circuit configured for use with a physical parameter measuring device, said application specific integrated circuit comprising:
(A) a single substrate material to which are coupled:
(i) a physiologic sensor configured to sense a physiologic parameter data of an individual;
(ii) a receiver configured to receive incoming data;
(iii) a microprocessor configured to receive the physiologic parameter data from the physiologic sensor and the incoming data from the receiver; and
(iv) a transmitter configured to transmit at least one of the physiologic parameter data and incoming data;
wherein the physical parameter measuring device comprises a wearable patch and the integrated circuit is a component of the wearable patch.

17. The application specific integrated circuit of claim 16, wherein the physical parameter measuring device comprises a wearable physical parameter measuring device and the integrated circuit is a component of the wearable physical parameter measuring device.

18. The application specific integrated circuit of claim 16, wherein the physiologic sensor is configured to measure at least one of acceleration, PT-INR, blood glucose, stoke volume, respiration and air flow volume, body tissue state, bone state, pressure, physical movement, body fluid density, the individual's physical location or audible body sounds, or a combination thereof.

19. An ECG monitoring device comprising
(A) an electrode configured to sense an electric potential on a first skin surface of a subject;
(B) a wireless transmitter configured to transmit a first wireless signal to a computing device;
(C) a receiver configured to receive a second wireless signal from the computing device;
(D) a microprocessor coupled to the first electrode, the wireless transmitter, and the receiver; and
(E) a non-transitory computer readable storage medium encoded with a computer program including instructions executable by the microprocessor that cause the microprocessor to:
(i) transmit the wireless signal with the wireless transmitter to the computing device, and
(ii) execute an instruction associated with the second wireless signal that is received by the receiver from the computing device.

20. The ECG monitoring device of claim 19, wherein the first wireless signal comprises an instruction executable by the microprocessor configured to cause the microprocessor to activate the electrode.

21. The ECG monitoring device of claim 19, wherein the incoming data comprises an instruction executable by the microprocessor configured to cause the microprocessor to transmit the second wireless signal to the computing device using the transmitter.

22. The ECG monitoring device of claim 19, wherein the incoming data comprises an instruction executable by the microprocessor configured to cause the microprocessor to analyze the electrical potential.

23. The ECG monitoring device of claim 19, wherein the transmitter comprises a Bluetooth transmitter.

24. The ECG monitoring device of claim 19, wherein the transmitter comprises a WiFi transmitter.

25. A handheld ECG monitoring device comprising:
(A) a first electrode configured to sense a first electrical potential sensed from a skin surface of an individual;
(B) a second electrode configured to sense a second electrical potential sensed from a skin surface of an individual;
(C) an application specific integrated circuit comprising:
(i) a single substrate material to which are coupled:
(a) a receiver configured to receive incoming data;
(b) a microprocessor configured to receive the cardiac parameter data from the cardiovascular monitoring sensor and the incoming data from the receiver; and
(c) a transmitter configured to transmit at least one of the cardiac parameter data and incoming data.

26. The ECG monitoring device of claim 25, wherein the incoming data comprises an instruction executable by the microprocessor configured to cause the microprocessor to activate at least one of the first electrode and the second electrode.

27. The ECG monitoring device of claim 25, wherein the incoming data comprises an instruction executable by the microprocessor configured to cause the microprocessor to transmit the first and the second electrical potential to a computing device using the transmitter.

28. The ECG monitoring device of claim 25, wherein the incoming data comprises an instruction executable by the microprocessor configured to cause the microprocessor to analyze the first and the second electrical potential.

29. The ECG monitoring device of claim 25, wherein the transmitter comprises a Bluetooth transmitter.

30. The ECG monitoring device of claim 25, wherein the transmitter comprises a WiFi transmitter.

31. A method for measuring physical parameters of an individual comprising:
(A) receiving a device comprising a sensor, a microprocessor, a receiver, and a transmitter;
(B) receiving a first signal with said receiver comprising an instruction;
(C) carrying out said instruction using said microprocessor;
(D) sensing a cardiovascular parameter; and
(E) transmitting the cardiovascular parameter using the transmitter.

32. The method of claim 31, wherein the sensor, the microprocessor, the receiver, and the transmitter are positioned on a single substrate.

33. The method of claim 31, wherein the instruction causes the microprocessor to activate the sensor.

34. The method of claim 31, wherein the instruction causes the microprocessor to transmit the physical parameter using the transmitter.

35. A method for analyzing and aggregating physical parameters of an individual comprising:
(A) receiving a device comprising a single substrate that comprises a sensor, a microprocessor, a receiver, and a transmitter;
(B) constructing an individual-based physiological baseline using data aggregated by a machine learning algorithm, wherein the data comprises sensor data, historical data, task-based data, and behavior based data;
(C) aggregating and representing a baseline data using the machine learning algorithm;
(D) analyzing and detecting abnormalities using the baseline data;

36. The method of claim 35, wherein the baseline data is constructed using demographic data of the individual.

37. The method of claim 35, wherein the baseline data is constructed using the sensor data when the individual performs pre-defined activities.

38. The method of claim 37, wherein the pre-defined activities include sleeping, sitting still, exercising, and walking.

39. The method of claim 35, wherein the baseline data is constructed using the sensor data when the individual performs routine tasks.

40. The method of claim 35, wherein the baseline data is represented in a multilayer neural network.

41. The method of claim 35, wherein the baseline data is represented in a multi-variant hyper space.

42. The method of claim 35, wherein the sensor data is analyzed using a multi-layer neural network classifier wherein the data is categorized into activity data and behavioral data

43. The method of claim 35, wherein the sensor data is analyzed using a supported vector machine classifier wherein the data is categorized into activity data and behavioral data

44. The method of claim 35, wherein the abnormalities are detected using a multi-layer neural network

45. The method of claim 35, wherein the abnormalities are detected using a supported vector machine classifier
